(19) United States
(12) Patent Application Publication
(10) Pub. No.: US 2014/0134716 A1
(43) Pub. Date: May 15, 2014

(54) Tissue Culture Device

(71) Applicant: Pioneer Hi-Bred International, Inc., Johnston, IA (US)

(72) Inventors: Steven M. Becker, Johnston, IA (US); Sam Ellis, Des Moines, IA (US); Yinghong Li, Urbandale, IA (US); Igor C. Oliveira, Johnston, IA (US)

(21) Appl. No.: 14/160,317

(22) Filed: Jan. 21, 2014

Related U.S. Application Data
(62) Division of application No. 12/650,010, filed on Dec. 30, 2009, now abandoned.
(60) Provisional application No. 61/143,892, filed on Jan. 12, 2009.

(51) Int. Cl. C12M 1/00

(52) U.S. Cl. CPC C12M 47/02
USPC 435/297.5

(57) ABSTRACT

A tissue culture device includes a container having one or more upstanding walls extending upwardly from a floor. The floor of the container has a media having nutrients or growth substances therein. A plurality of plant tissues are within the container compartment and are placed upon a screen between the plant tissues and the media. The screen is tamped downwardly onto the media so that the plant tissues can get nutrients from the media, and so that waste products are transferred into the media. The screen is also removable through the open end of the container so as to remove all of the plurality of the plant tissues from the container at once. The plant tissue device can then be placed in another container having any cultured media as needed for the correct maintenance, propagation and development of plant tissue in culture.

### RELATED APPLICATIONS

This application claims priority under 35 U.S.C. §119 to U.S. Provisional Patent Application No. 61/143,892 filed Jan. 12, 2009, which application is hereby incorporated by reference in its entirety.

### FIELD OF THE INVENTION

The present invention relates to apparatuses and methods for a tissue culture device.

### BACKGROUND OF THE INVENTION

Cellular tissues used in the biological industry are commonly cultured in nutrient media and placed in tissue culture vessel (e.g. Petri dishes) for growth, maintenance and differentiation.

According to many current methods of culturing biological tissue on various types of media, the tissue is placed in direct contact with the media. Sometimes several samples of biological tissues are placed in a tissue culture vessel on the media. The progression of the tissue culture routine often demands that the tissue in question be transferred to a new tissue culture vessel. For instance, one may desire or need to transfer the tissue culture to a new media to continue providing the tissue with fresh nutrients or to move the tissue to a different media in order to expose the tissue to substances needed for the tissue culture process (i.e., hormones, salt, metals, etc.). In addition, media and the tissue culture vessel can develop a buildup of waste materials generated by the growing tissue. Therefore, it is desirable to remove the growing tissues from the original tissue culture vessel, and place them in contact with another media within another tissue culture vessel. This is a time consuming task, and involves individual removal of the biological tissue with tweezers or forceps. A device that permits multiple tissue samples to be removed in a single step as opposed to individual steps will result in considerable savings in time expense and resources, whether human, mechanical or otherwise.

Therefore, a primary embodiment of the present invention is the provision of a tissue culture transfer device that permits the removal of a plurality of biological tissue as a group from one tissue culture vessel to another, thereby bypassing the need for moving the individual biological tissues one at a time.

A further embodiment of the present invention is the provision of a tissue transfer device which utilizes a screen device which may be tamped down onto the media so that the media or nutrients coming from that media will be engaged in the perforations in the screen device and therefore come in contact with the tissues which are on top of the screen.

A further embodiment of the present invention is the provision of a tool which will permit removal of the screen device and replacement of the screen device within another tissue culture vessel or container, for instance a Petri dish, without jeopardizing the culture process or plant tissues.

A further embodiment of the present invention is the removal of a plurality of biological tissues from a tissue culture vessel or container in a singular motion and the replacement of those plant tissues in a singular motion in another tissue culture vessel or container.

Another embodiment of the present invention is the provision of a media device which is simple in operation, which is easy to operate, and which shortens the time necessary to remove biological tissue from one tissue culture vessel or container to another.

### BRIEF SUMMARY OF THE INVENTION

For the purposes of this disclosure, it is to be understood that biological tissues (also referred to as tissue or tissues) may include any form of biological tissues to be grown in tissue culture. Such tissues may include, but are not limited to, plant tissues, mammalian tissues, insect tissues, fungal tissues, and other types of tissues. Within such groups of tissues, various types of tissues may be cultured. For example, plant tissue cultures may be performed with tissues including, but not limited to, embryo tissue, stem tip tissue, node tissue, meristematic tissue, root tissue, etc.

The culturing device of the present invention in one aspect may include a membrane having a perimeter and a reinforcing member extending around the perimeter to support the membrane, a top surface for supporting a biological material thereon, and a plurality of openings that permits contact between a medium and the biological material so that the biological material can obtain desired growth substances from the medium. The membrane is movable so as to remove the biological material all at once from the medium.

According to another feature of the invention, the reinforcing member may include at least one attachment point to provide movement and/or handling the membrane without harming or contaminating the biological material thereon.

According to another feature of the present invention, the culturing device may include a tool having an attachment device for cooperatively engaging and releasing the at least one attachment point to provide movement and/or handling of the membrane.

According to another feature of the present invention, the attachment device may include an aperture for cooperatively engaging and releasing the attachment point.

The present invention may include a tissue culture vessel such as a container having a floor and one or more upstanding walls extending upwardly from the floor. The container optionally includes a top that is removable defining a container compartment therein. The top is removable to leave an open end therein. A container with a top is usually desirable in tissue culture methods because of the need for sterility, however, there may be times when a top is not used, or when a container has a temporary cover made of foil or other materials, rather than a formed top or lid. A culture medium is placed within the container compartment and the culture medium includes growth substances, such as, for example, nutrients, therein. A plurality of biological tissue is within the container compartment, and is placed on a screen, the screen being located between the plurality of biological tissues and the culture medium. The screen permits contact between the culture medium and the biological tissues so that the tissues can obtain nutrients and any other desired growth substances from the medium. The screen is removable through the open end of the container so as to remove all of the plurality of biological tissues from the container at once.

According to another feature of the invention, the screen may include one or more attachment points in which case a tool is attachable to the attachment point for removal of the screen through the open end of the container and out of the container compartment.

According to another feature of the present invention, the tool includes a tamping apparatus for tamping the screen downwardly toward the medium so that the media is in contact with a plurality of tissues.

According to another feature of the present invention, the tool includes a tamping apparatus with a lip portion for manipulating the screen and/or calli.

According to another feature of the present invention, the screen includes a plurality of openings therein and the medium and/or its components are capable of passing through the openings to contact the tissues.

According to another feature of the present invention, the screen includes a perimeter and a reinforcing member extending around the perimeter.

According to another feature of the present invention, the screen is removable from the first mentioned container and is moveable into contact with a second medium within a second container having a second floor and one or more upstanding walls extending upwardly from the floor to create a second container compartment.

The culturing device of the present invention in another aspect may include a screen having a top surface for supporting a biological material thereon, a plurality of openings that permits contact between a medium and the biological material so that the biological material can obtain desired growth substances from the medium, and the screen may be movable so as to remove the biological material all at once from the medium. The culturing device also includes a tool having a tamping portion for tamping the screen downwardly toward the medium to urge the biological material into contact with the medium and an attachment portion for cooperatively holding and releasing the screen to provide movement and/or handling of the screen.

According to another feature of the present invention, the screen includes at least one attachment point to provide movement and/or handling of the screen.

According to another feature of the present invention, the attachment portion on the tool cooperatively engages and disengages from the attachment point on the screen to provide movement and/or handling of the screen using the tool.

The culturing device of the present invention in yet another aspect may include a tool adapted for use with a screen that permits contact between a medium and a biological material so that the biological material can obtain desired growth substances from the medium. The tool comprises a handle having gripping means for manipulating the tool. The handle terminates in a tamping portion having a generally planar surface, alone or in combination with a lip portion, adapted for movement of biological materials into contact with a medium for providing growth substances to the biological material, The tool also has an attachment portion for cooperatively holding and releasing the screen to provide movement and/or handling of the screen so as to remove the biological material all at once from the medium.

According to another feature of the present invention, the gripping means comprises at least one of a thumb plate opposite the tamping portion on the handle, a cross bar spaced between the thumb plate and the tamping portion on the handle, and/or an appendage on the handle having a geometry to provide for movement and/or handling of the tool.

According to another feature of the present invention, the attachment portion comprises an aperture or other geometry in the tamping portion of the tool or disposed at a terminal end of the tool adapted for engaging, gripping and releasing an upstanding member or apertures associated with the screen to move the screen and biological material all at once.

The foregoing embodiments may also be achieved from one or more methods of the present invention. According to one aspect of the present invention, a method for culturing biological tissue is disclosed. The method includes the steps of taking a membrane having a supporting perimeter and a top surface adapted for supporting biological tissue thereon, mounting a plurality of biological tissues on the top surface of the membrane, inserting the membrane having the plurality of biological tissues on the top surface thereof into contact with a medium so that at least a portion of the medium passes through the membrane and engages the biological tissues, and removing the plurality of plant tissues at once from the medium by removing the membrane from the medium.

According to another feature of the present invention, the supporting perimeter of the membrane is handled for inserting and/or removing the membrane from the medium.

Another method utilizes a tissue culture vessel such as a container having a floor and at least one upstanding wall extending from the floor to create a container compartment therein. A culture medium is placed within the container compartment and the culture medium includes nutrients therein. A plurality of tissues are mounted on the top surface of a single screen. The screen is inserted with a plurality of tissues on the top surface thereof into the container compartment so that a bottom surface of the screen contacts the culture medium. The culture medium and/or its components can then engage the tissues by either passing through the screen and/or having the culture medium in direct contact with the tissues. The screen is removed from the container compartment after a period of time with the plurality of tissues contained thereon. In certain instances there is actually contact between the tissues and the culture medium.

According to another feature of the present invention, the screen is tamped downwardly into contact with the culture medium so that the growth substances in the medium pass through the screen and engage the tissue,

According to another feature of the present invention, the screen includes a plurality of apertures therein and the tamping downwardly step includes pressing the screen against the culture medium so that the medium and/or its components can then engage the plant tissue by either passing through the screen and/or having the medium in direct contact with the biological tissue.

According to another feature of the present invention, the tool has an attachment device, and the step of removing the screen from the container compartment comprises attaching the attachment device to the screen and using the tool or forceps to remove the screen from the container.

According to another feature of the present invention, the screen or an attachment point on the screen is engaged with a tool that removes and lifts the screen away from the medium and container compartment with the tissues thereon.

### BRIEF DESCRIPTION OF THE DRAWINGS

FIG. 1A is a perspective view of the present device.

FIG. 1B is a perspective view of the present device according to another aspect of this invention.

FIG. 2 is a sectional view showing the tamping of the screen downwardly.

FIG. 3 is a sectional view showing the removal of the screen from the open end of the container.

FIG. 4 is a sectional view similar to FIG. 3 showing the lid on the container with the biological tissues and the screen and the medium therein.

FIG. 5 is a sectional view taken along line 5-5 of FIG. 2.

FIG. 6 is a perspective view of a tool of the present invention.

FIG. 7 is a perspective view of a screen of the present invention.

FIG. 8 is a perspective view of another screen of the present invention.

### DETAILED DESCRIPTION

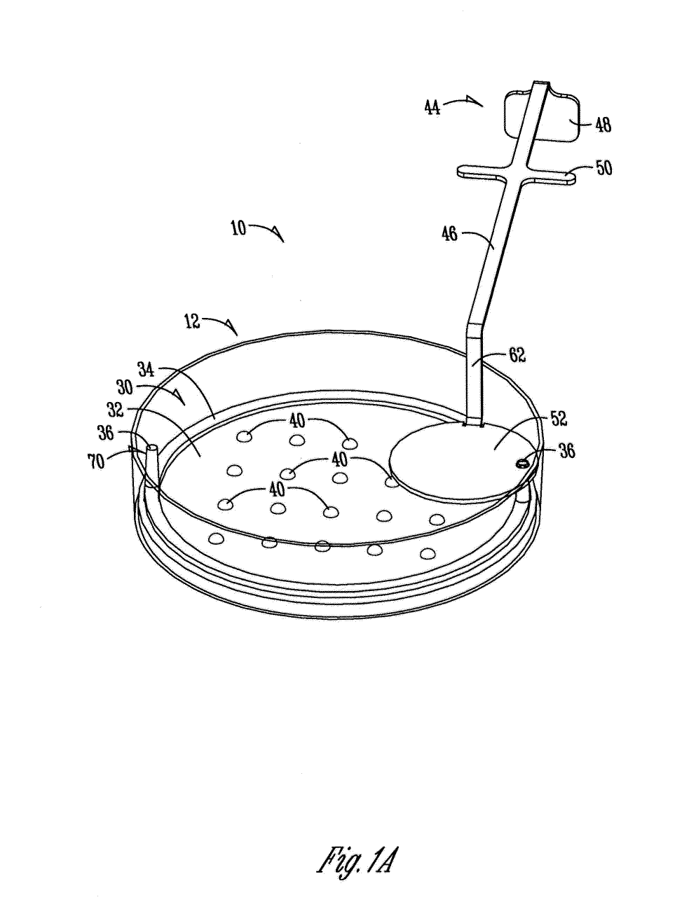

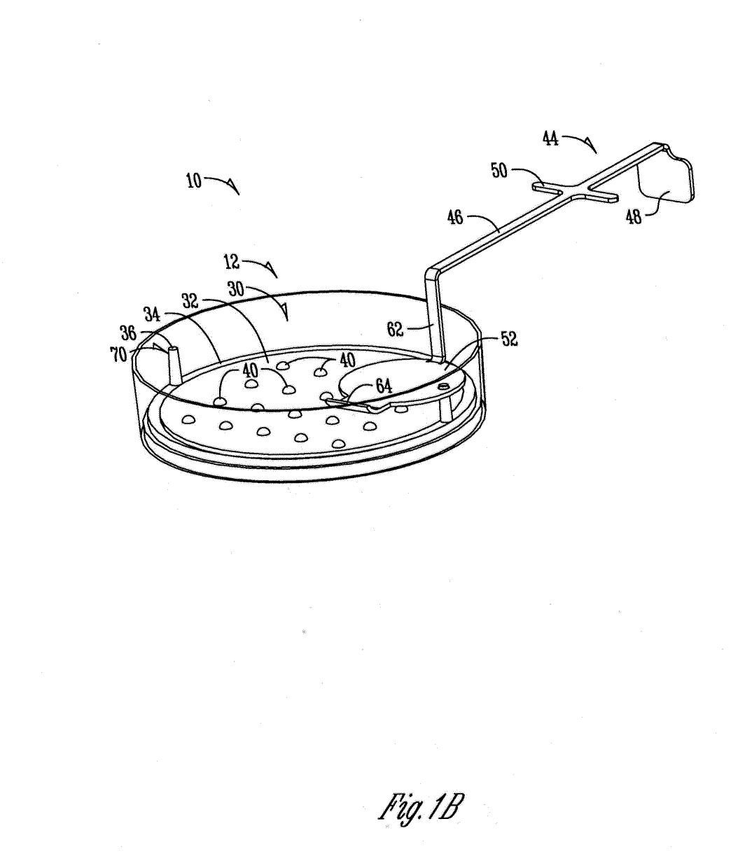

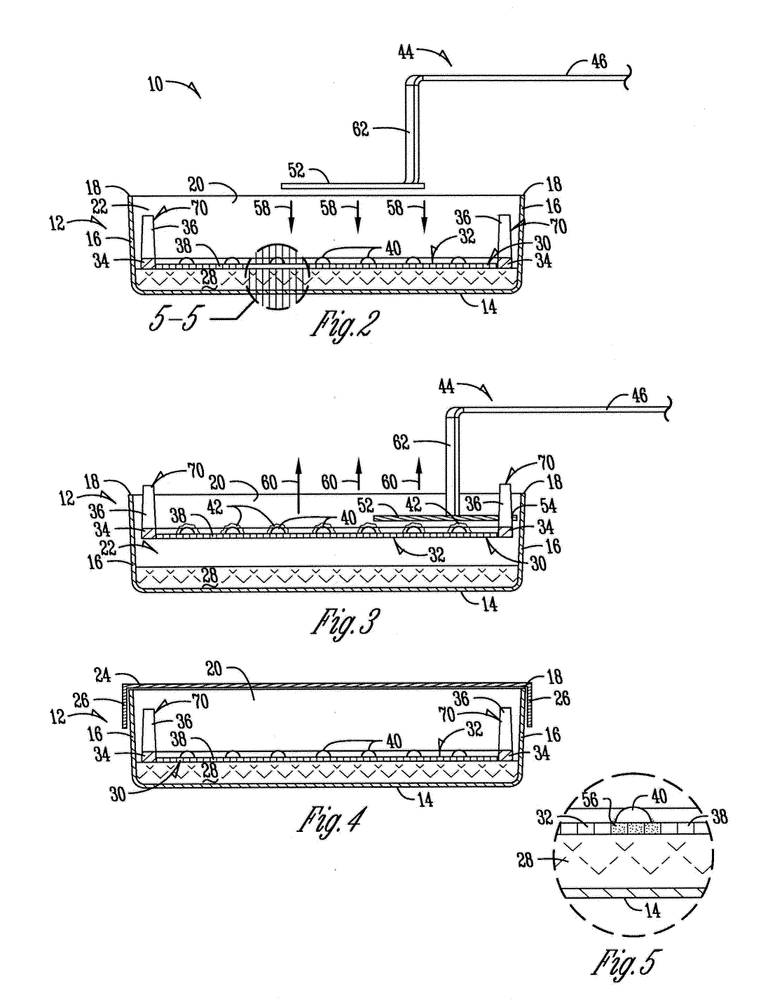

The numeral 10 generally designates a tool utilized with a tissue culture vessel, such as Petri dish or container 12. Container 12 includes a floor 14 (FIGS. 2-4) and an upstanding wall 16. The wall 16 may be a singular wall as shown in FIG. 1A-B, or it may be a plurality of wails in the irregular configuration of a square or other polygon. The top edges of upstanding walls 16 are shown at 18. These top edges 18 form an open top 20. The container optionally includes a container compartment 22 and a lid or top 24 (FIG. 4). The lid 24 may include lid sidewalls 26.

Inserted within the container compartment 22 is a medium 28 which includes nutrients or growth materials which are absorbed into the biological tissues to be grown. The medium is a gel-like substance, and includes a plurality of nutrients or a single nutrient depending upon particular growth desired and may also include selective agents, hormones, and other substances. Examples of suitable mediums 28 include various Agarose concentrations such as, but not limited to, 8 g/L, 6 g/L, 4 g/L, 2.4 g/L and 1.2 g/L. The 4 g/L concentration of Agarose used in conjunction with a Nitex Nylon mesh type 06-1000/57 (Sefar Filtration Inc.) was identified through testing as providing the most prolific callus. Different nutrients may be utilized in different gels or media for different purposes of tissue culture. These purposes are all known to those skilled in the art. Sometimes the medium becomes exhausted of growth substances, such as nutrients, therein. As the tissue begins to grow, it will form a mass of tissue called callus. The tissues or calli can also develop a buildup of waste products as they grow. In these cases, it is sometimes desirable to remove the tissues 40 or the calli 42.

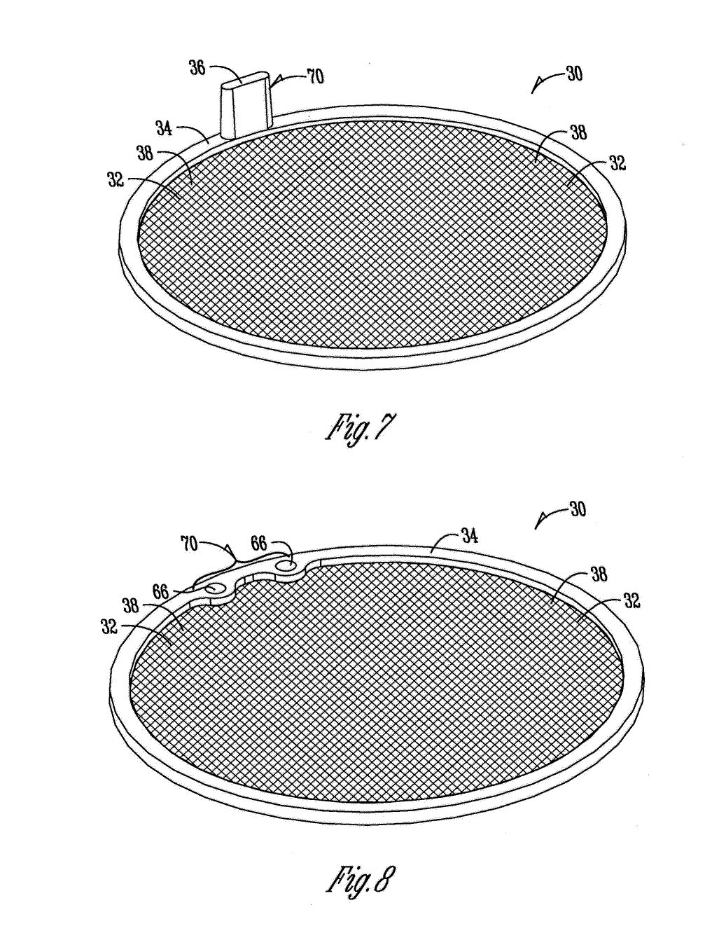

A screen 30 includes a screen material 32 and a rim 34 around the perimeter thereof. An attachment point 70 is configured in the rim, and in one aspect of this invention extend upwardly therefrom as a pair of upstanding attachment members 36 as shown in FIGS. 1A-4. FIG. 7 illustrates the attachment point 70 as a single upstanding member 36 attached to the rim. FIG. 8 illustrates the attachment point 70 as a pair of apertures 66 configured in the rim. A tool 44 as further described below is capable of attachment to attachment points 70. Other tools not shown, such as forceps, may be used to interface with the attachment point 70 for manipulating the screen 30, such as a tool adapted to grip the attachment member(s) 36 or adapted to be inserted into the pair of apertures 66.

The screen material 32 includes a plurality of apertures 38. The choice of screen material 32 is highly dependent on the ability of the material to permit sufficient contact between callus and the media, nutrient update and waste removal. Preferable screen materials 32 include various Nitex Nylon mesh types, such as mesh type 06-1000/57, provided by Sefar Filtration Inc., 111 Calumet Street, Depew, N.Y. 14043.

This invention contemplates additional embodiments for the membrane 36, such as for example a membrane having a perimeter without a reinforcing member, a membrane with a support system within the perimeter of the membrane, a membrane having sufficient rigidity to support its own weight and any biological material, and interface points associated with the membrane and/or reinforcing member adapted to allow movement to be imparted to the membrane.

Mounted on screen material 32 are a plurality of biological tissues 40. These tissues receive nutrients from the medium 28 and begin growth. The growth portion of the tissues is referred to as callus 42,

A tool 44 (FIGS. 1A-3 and 6) is shown having a handle end 46 comprising a hoe-shaped end 48 and a cross bar 50. The hoe-shaped end 48 and the cross bar 50 are intended to accommodate a person's hand, and may be varied depending upon the particular tool used and the particular function desired. In one form of use, the cross bar 50 may provide support for hooking the index finger and middle finger of a user and the hoe-shaped end 48 may provide support for the thumb of the user. At the opposite end of the tool 44 is a tamping end 52 which may be varied in shape and size, In FIG. 1A, the tamping end 52 of the tool 44 is generally circular in shape. In FIG. 6, the tamping end 52 of the tool 44 is generally rectangular in shape. The tamping end 52 is offset from the handle end 46 and placed at an angle with respect to the offsetting portion 62 of the tool 44 so that the tamping end 52 may be tamped downwardly on the screen material 32. The tamping end 52 and handle end 46 of the tool 44 lie preferably in parallel planes. Other embodiments of the tool 44 include the tamping end 52 and handle end 46 of the tool 44 residing in non-parallel planes. In another aspect of this invention, offsetting the tamping end 52 from the handle end 46 allows the user to keep the tamping end 52 parallel with the screen material 32. The present invention contemplates that the length of the offset portion 62 could be altered to accommodate container walls of varying height. This could include providing an adjustable offsetting portion 62 on the tool 44 to allow the offsetting portion 62 to be adjusted in length depending upon the depth of the container 12 or the height of the container wall. An adjustable offsetting portion 62 or varied lengths of the offsetting portion 62 would allow the user to keep the tamping end 52 parallel with the screen material 32. This is important because tamping places the medium 28 in direct contact with the screen material 32 and causes portions 56 of the medium 28 to extend upwardly through apertures 38 in screen material 32. This permits the tissues 40 to contact the portions 56 of the medium 28 and receive nutrients therefrom, even though the screen material 32 is positioned therebetween. The tamping of the screen material 32 downwardly is a useful function of the present invention.

A lip portion 64 is shown in FIGS. 2B and 6 extending from the tamping end 52 of the tool 44. The lip portion 64 slopes gradually upward to provide an upward sloping bottom surface for manipulating the screen material 32 or calli 42. For example, the lip portion 64 is shaped having a natural curvature for pressing down the callous 42 into the media 28 as the tool 44 is drawn across the media 28. An edge 68 of the lip portion 64 may be tapered to allow for easier transition of calli 42 onto the lip portion 64 and/or tamping end 52 of the tool 44 to allow calli 42 to be moved, removed or repositioned.

A hole 54 is shown in tamping end 52 and is capable of surrounding the upstanding attachment member(s) 36 of screen 30 as is readily shown in FIG. 3. Thus, in the case where the attachment point 70 comprises an upstanding attachment member 36, it is possible to attach the tool 44 by extending the upstanding attachment members 36 through the aperture or hole 34 and by lifting the tool 44 to lift the screen 30 upwardly. The hole 54 in the tamping end 52 may be oversized relative to the upstanding attachment members 36 to allow for easier insertion of the upstanding attachment members 36 into the hole 54. An oversized hole 34 (relative to the diameter of the upstanding attachment members 36) would also assist in preventing the two mating surfaces of the hole 54 and the upstanding attachment members 36 from binding up when it is desired that the tool 44 be separated from the screen 30. Providing an oversized hole 54 would also allow the user to pitch the tool 44 at an angle relative to the screen 30 such that the mating surfaces of the hole 54 and the upstanding attachment member 36 bind together for movement of the screen 30 with the tool 44 and unbind from each other when the tamping end 52 of the tool 44 is brought back to a position substantially parallel with the screen 30 to allow the tool 44 to be separated from the screen 30. The above configuration allows the user to move the screen 30 using the tool 44 and doesn't necessitate the user to handle, touch or pry the screen 30 from the tool 44 when the user desires to separate the screen 30 from the tool 44. The present invention contemplates that the size of the hole 54 could be altered to accommodate various sizes and shapes of attachment points 70. For example, the hole 54 in the tamping end 52 of the tool 44 may be sized to receive the upstanding attachment member 36 illustrated in FIG. 7. Other tools, such as forceps, may be used to grip the attachment point 70, including gripping the upstanding attachment member 36 in FIG. 7 or gripping the pair of apertures 66 in the perimeter 34 of screen 30 illustrated in FIG. 8.

The method of operation is as follows: First, a media 28 is placed within the container compartment 22 adjacent the floor 14 thereof, Media 28 is a gel-like substance which includes one or more nutrients or growth substances capable of supporting life. The growth substances are preferably those appropriate for the particular biological tissue to be grown in the container. Next, a plurality of biological tissues 40 are placed on the screen material 32. Next, the screen 30, having the plurality of biological tissues 40 thereon, is inserted into the Petri dish or container 12. The biological tissues 40 may be 20 or more, but the particular number of biological tissues is not important to the invention. The primary requirement is that a plurality of biological tissues 40 are placed on the screen material 32. Also, it is important that the screen 30 have a diameter which is slightly smaller than the diameter of container 12 so that screen 30 covers the media 28 while at the same time the screen 30 is removable through open end 20.

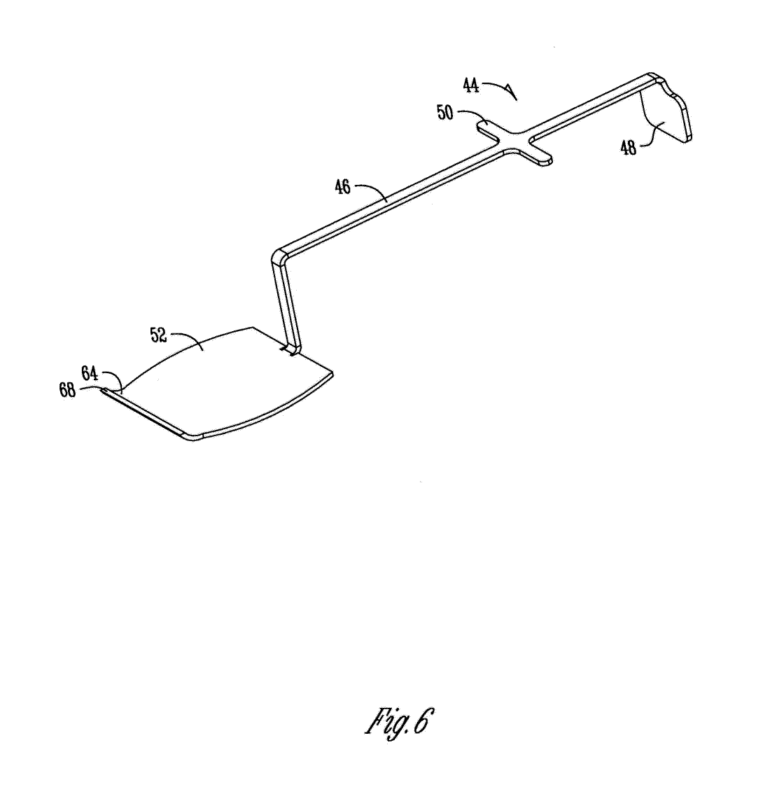

Next, the operator tamps the screen 30 downwardly into contact with the media 28. The biological tissues 40 are preferably not damaged by the use of the tamping end 52 of the tool 44. The primary function of the tamping process is that the screen material 32 must be tamped downwardly into contact with the medium 28. As a result, the particles of medium 56 as shown in FIG. 5 extend upwardly through the apertures 38 in screen material 32 so as to engage the biological tissues 40. The biological tissues 40 receive the nutrients from the media 28 and begin to grow. Preferably the lid or top 24 is placed over the upstanding walls 16 and the entire container 12 is placed in a refrigerated condition. The biological tissues 40 begin to grow and calli 42 are formed.

Sometimes the nutrients in medium 28 are exhausted and therefore it is desirable to remove the screen from the container 12 and place it in a second container having upstanding walls and having a new medium 28 therein. Sometimes, it is desirable to transfer the biological tissues/calli to different nutrients in the medium 28. In both of these cases, it is desirable to lift the screen 30 out of the container 12 and into a new container. This is accomplished by placing the hole 54 over the attachment point 70 configured as upstanding attachment members 36 and by lifting the screen 30 outwardly through the open end 20 as shown in FIG. 3.

Prior methods of removal of the biological tissues 40 involved forceps that were applied to each of the biological tissues 40 or calli 42 separately. This was a time consuming task, and sometimes resulted in damage of the biological tissues 40 or the callus 42. The present invention accomplishes this by lifting the entire screen 30 upwardly through the opening 20, and permitting a plurality of the biological tissues 40 or calli 42 to be lifted upwardly as a group rather than individually as with forceps, Furthermore, there is no damage to the biological tissues 40 or the callus 42.

The invention has been shown and described above with the preferred embodiments, and it is understood that many modifications, substitutions, and additions may be made which are within the intended spirit and scope of the invention. From the foregoing, it can be seen that the present invention accomplishes at least all of its stated objectives.

1.-16. (canceled)

17. A culturing device comprising:
a screen having:
a. a top surface for supporting a biological material thereon;
b. a plurality of openings that permits contact between a medium and the biological material so that the biological material can obtain desired growth substances from the medium; and
c. the screen being movable so as to remove the biological material all at once from the medium.
a tool having:
a. a tamping portion for tamping the screen downwardly toward the medium to urge the biological material into contact with the medium;
b. a lip portion having a surface for manipulating the biological material; and
c. an attachment portion for cooperatively holding and releasing the screen to provide movement and/or handling of the screen.

18. The culturing device of claim 17 wherein the screen further comprises at least one attachment point to provide movement and/or handling of the screen, the attachment point comprising at least one upstanding attachment member.

19. The culture device according to claim 18 wherein the attachment portion on the tool comprises an aperture, the aperture cooperatively engages and disengages from the upstanding attachment member on the screen to provide movement and/or handling of the screen using the tool.

20-32. (canceled)
